(19) United States
(12) Patent Application Publication
(10) Pub. No.: US 2024/0416140 A1
(43) Pub. Date: Dec. 19, 2024

(54) SYSTEM AND METHOD FOR ADMINISTERING PULSE TRAINS TO ALTER CIRCADIAN RHYTHMS

(71) Applicant: KORRUS, INC., Los Angeles, CA (US)

(72) Inventors: Benjamin Harrison, Los Angeles, CA (US); Paul Kenneth Pickard, Los Angeles, CA (US)

(21) Appl. No.: 18/813,260

(22) Filed: Aug. 23, 2024

Related U.S. Application Data
(63) Continuation of application No. PCT/US2023/013676, filed on Feb. 23, 2023.
(60) Provisional application No. 63/312,906, filed on Feb. 23, 2022.

(51) Int. Cl. A61N 5/06

(52) U.S. Cl. CPC A61N 5/0613; A61N 2005/0626; A61N 2005/0632

(57) ABSTRACT

A system of adjusting the phase of a user's circadian rhythms, said system comprising: a high EML light source configured for delivering pulsed high EML light in a pulsed mode of operation, wherein the pulsed high EML light has a frequency and intensity sufficient to effect circadian phase adjustment in said user, and secondary functionality comprising transmitting electromagnetic radiation (EMR) to said user.

### REFERENCE TO APPLICATION

This application is based on U.S. Provisional Application No. 63/312,906, filed Feb. 23, 2022, which is incorporated herein by reference.

### FIELD OF INVENTION

This invention relates generally to light therapy, and, more specifically, to a method and system for administering pulse trains to alter a user's circadian rhythms.

### BACKGROUND

Sunrise lamps have become popular to help people wake up. Sunrise lamps function as alarm clocks by providing light in a manner that simulates light from the sun in the morning. Because sunrise varies from day to day, not only in time, but also in intensity, owing to, for example, atmospheric and weather related phenomena, providing simulated morning sunlight at a consistent time and intensity helps as a circadian stabilizer. However, for individuals with a late chronotype, the benefits of providing a consistent simulated sunrise may be partially offset by the lack of synchronicity between the individual's circadian rhythm and the operation of the sunrise lamp. Thus, a late riser will consistently experience a feeling of being tired and unprepared to wake up when the sunrise lamp activates.

Applicant recognizes that this shortcoming in not limited to sunrise lamps. Any light therapy that attempts to regulate circadian rhythm may be bucking the individual's inherent circadian clock. The result is a superficial adjustment of a person's alertness that is at odds with the person's true circadian rhythm-thus, while the person may be awake, he or she is still tired. What is needed is a system that synchronizes a person's inherent circadian rhythm with a circadian stimulation. The present invention fulfills this need among others.

### SUMMARY

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

Applicant recognizes a person's inherent circadian rhythm may be synchronized with that of a circadian stimulation lighting system by providing a pulse train of relatively high EML light. These light pulse trains are known to elicit circadian phase adjustment without disrupting sleep architecture. Applicant therefore discloses herein combining these high EML pulse trains with other systems configured to moderate or monitor a person's awareness.

Applicant further recognizes that integrating a system for delivering EML pulses with a light therapy system or other system for moderating or monitoring a person's awareness realizes a number of synergies. First, combining EML pulse trains with a light therapy device for moderating or monitoring a person's awareness improves the effectiveness of the light therapy application by synchronizing a person's inherent circadian cycle/rhythm with the desired circadian stimulation of the light therapy.

Second, integrating an EML pulse system with a system having secondary functionality, such as light therapy, increases the usability of the system. More specifically, Applicant recognizes that a user is unlikely to use a system which is dedicated to providing solely EML pulses. For example, a user is unlikely to be compliant long term with a pulse train system that must be set up and worn by the user, as in the prior art. Rather, in a preferred embodiment, the user receives the pulses subconsciously or even unconsciously (e.g., during sleep). To this end, Applicant proposes a system that provides the user with a secondary functionality which drives the user's use of the system, and, thus, consequently the use of the EML pulse system too.

Third, integrating an EML pulse system with a system having secondary functionality, such as light therapy, increases the efficiency/manufacturability of the system. In all likelihood, the EML pulse system and the system providing secondary functionality will share common components—e.g., high EML LEDs, LED drivers, power supplies, processors, etc. Integrating these systems in a common system allows these common components to be shared.

Generally, although not necessarily, this secondary functionality will involve transmitting electromagnetic radiation (EMR) to the user. The EMR may take the form of, for example, visible light, invisible light (e.g., infrared), or even radio frequency waves. The secondary functionality may vary according to the application. For example, in one embodiment, the secondary functionality might be lighting-either specialty lighting (e.g., light therapy, sunrise lamp, or airline cabin lights), general lighting (e.g., office lighting, or home lighting), or displays (e.g., smartphone displays, computer monitor, or televisions). In another embodiment, the secondary functionality may involve, for example, sensing functionality (e.g., occupancy sensors, or vehicle eye monitoring sensors). Still other secondary functionalities will be obvious to those of skill in the art in light of this disclosure.

Accordingly, in one embodiment, the system comprises: (a) a high EML light source configured for delivering pulsed high EML light in a pulsed mode of operation, wherein the pulsed high EML light has a frequency and intensity sufficient to effect circadian phase adjustment in said user; and (b) secondary functionality for transmitting electromagnetic radiation (EMR) to said user.

Yet another independent aspect of the present invention is the acquisition of data to automatically “dose” the EML pulses. Accordingly, in one embodiment, the system comprises is configured for (a) receiving one or more sensor inputs indicative of at least one of a circadian rhythm of an individual, an environmental attribute and a location attribute; (b) determining based, at least in part, on the received one or more sensor inputs a pulse train schedule for the individual the pulse train schedule; and (c) emitting light from the high EML light source at the individual in accordance with the pulse train schedule, wherein the pulse train schedule comprises a description of a timing and a description of each of a plurality of light pulses emitted by the high EML light source sufficient to produce a predetermined change in the circadian rhythm of the individual.

### BRIEF DESCRIPTION OF FIGURES

FIG. 1 shows one embodiment of the system of the present invention.

### DETAILED DESCRIPTION

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

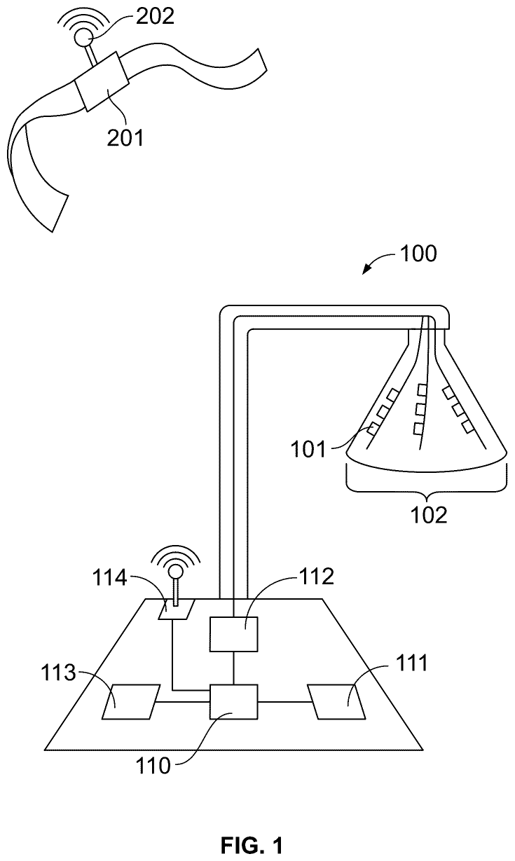

Referring to FIG. 1, one embodiment of the system 100 is shown. In this embodiment, system 100 comprises: (a) a high EML light source 101 configured for delivering pulsed high EML light in a pulsed mode of operation, wherein the pulsed high EML light has a frequency and intensity to effect circadian phase adjustment in a user; and (b) secondary functionality 102 for transmitting electromagnetic radiation (EMR) to said user. In this particular embodiment, the system 100 is a sunrise lamp integrated with a system for delivering pulsed EML light. Each of these elements is described in greater detail below and in connection to selected alternative embodiments.

Using pulse high EML light to adjust circadian phase in a user is known. For example, suitable results have been achieved using a light having a peak wavelength of about 485 nm, a frequency of 1/7.5 Hz (or 0.133 Hz), and a pulse duration of 2 ms. For additional information, see RAYMOND P. NAJJAR, JAMIE M. ZEITZER, Temporal Integration of Light Flashes by the Human Circadian System, J Clin Invest. 2016; 126 (3): 938-947, hereby incorporated by reference.

The high EML pulse light system may be integrated with a variety of different systems for providing secondary functionality. In one embodiment, the secondary functionality relates to a lighting system. For example, in one particular embodiment, the high EML pulse train generator is integrated into a light therapy system, e.g., a sunrise lamp, to synergistically align an individual's circadian rhythm with a simulated sunrise awakening occurrence.

In another embodiment, the invention relates to a system that combines airline cabin lighting with a high EML pulse train generator to synergistically align an individual's circadian rhythm with the time zone of the airliner's destination or other schedule. It is typical, for example, for a plane flight to extend between different time zones. On overnight flights, such as “red eye” flights from the west cost of the US to the east coast of the US during the nighttime, the effects on an individual's circadian rhythm may be pronounced. In such instances, the interior cabin of a plane may pulse high EML light according to a predetermined or dynamically derived schedule so as to transition the circadian rhythms of the passengers to be more fully awake upon landing. In another example, a football team heading to the west coast for a game may be subjected to a pulse train schedule that will enhance the wake state of the players for the projected future game time.

In yet another embodiment, a circadian friendly display (e.g. computer display, TV, or smart phone) is integrated with a high EML pulse train.

Still other applications and synergies of using pulse lighting with other lighting systems will be obvious to those of skill he art in light of this invention

Although the present invention recognizes the synergies of combining EML pulsing with light therapy or other light delivery systems, the secondary functionality may be different from a light therapy or lighting systems in general. For example, in one embodiment, the secondary functionality involves monitoring the user. For example, in one embodiment, monitoring comprises sensing occupancy or user awareness. Sensing user occupancy is well known and involves, for example, monitoring infrared or reflective EMR signals, and monitoring user awareness (which is often used in vehicles to ensure the driver is awake/aware) generally, although not necessarily, involves monitoring the user's eye using infrared LEDs. In these applications, the sensing functionality can serve a dual function in sensing the presence or awareness of the user to ensure the dose of EML pulsing is receive.

In one embodiment, the high EML pulsing system is combined with two or more second functionalities.

In accordance with exemplary and non-limiting embodiments, an individual's circadian rhythm schedule may be defined by the individual or may be partially or completely computed or otherwise ascertained by the system based, at least in part, on sensor information and data. Referring to FIG. 1, one embodiment of the system 100 is shown, which uses sensor data to schedule high EML light pulses. Aside from the components identified above, the system has one or more processing devices 110, and a non-transitory memory device 111 in communication with the one or more processing devices, the non-transitory memory storing instructions that when executed by the one or more processing devices, result in the system 100: (a) receiving one or more sensor inputs from wearable sensor 201 indicative of at least one of a circadian rhythm of an individual, an environmental attribute, or a location attribute; (b) determining based, at least in part, on the received one or more sensor inputs a pulse train schedule; (c) emitting light from the high EML light source 101 at the user in accordance with the pulse train schedule, wherein the pulse train schedule includes the duration and time at which the pulsed EML light is delivered to a user to produce a predetermined shift in the phase of the user's circadian rhythm; and (d) activating said secondary functionality 102.

In this particular embodiment, the system also comprises a power supply 113, a wireless interface 114, and at least one driver for driving the high EML light source 101 and secondary functionality 102.

In some instances, the sensor data may be obtained from a wearable sensor 201 in close proximity to the individual. In other instances, the sensor may form a part of the system or may be in communication with the system, such as via Bluetooth or any other form of wireless interface 202 or wired communication.

For example, a wearable sensor may detect biometric data including, but not limited to, an individual's heartrate, respiration, temperature, mental acuity, drowsiness, etc. The data so collected may be stored in the sensor or transmitted to an external storage device including, but not limited, cloud storage. Data reflecting historical sensor data of the individual as well as historical sleep patterns, e.g., historical sleep and wake times and the like, may be stored for future access. Once collected and stored, the system may operate to determine a circadian rhythm or cycle for the individual and to compute or otherwise derive a pulse train schedule for the individual the execution of which may serve to alter the individual's circadian rhythm.

When computing the pulse train schedule, external parameters may be considered. For example, an individual packs his sunrise lamp for a trip and plugs it in facing his bed in a hotel in a city in a time zone with an hour difference from the city if his embarkation. When the individual goes to sleep, the system may sense, such as via facial recognition, the identity of the individual and may retrieve data indicative of the geographic location of the individual. The system may note, for example, that the individual tends to wake up every morning at 6:00 am. Noting that, with the time difference and in the present instance, the individual would tend to wake up around 7:00 am, the system may operate to pulse light at the individual while he sleeps in order to shift his circadian rhythm back by approximately one hour.

Once an appropriate schedule is determined, the system may operate to provide the pulses of high EML light in accordance with the schedule. In some embodiments, the system may monitor the environment surrounding the pulse train emitter and, in response thereto, may dynamically modify the pulse train schedule. For example, the system may utilize a thermal camera in communication with or forming a part of the system and determine that a temperature of an individual indicates that his temperature is not rising as would be expected if the individual's body was preparing to wake up. In response, the system may operate to hasten a change in the individual's circadian rhythm by, for example, altering the frequency, intensity, or wavelength of the pulse trains.

As noted, a pulse train may be derived based, at least in part, upon one or more manual configurations, received sensor data, historical data and external environmental data. In some instances, the system may query the individual. For example, before laying down to sleep, the individual may activate the system. Upon doing so, the system nay issue an audible query such as, “Good night. When do you wish to be awakened?” Based on the verbal response from the individual, the system may operate to formulate a schedule based on any or all of the above noted sources of data.

As described herein, embodiments of the system are drawn broadly to encompass any and all methods by which sensor data and other data may be gathered from an individual or the environment, stored, and retrieved by the system to enable determination of a pulse schedule. It is envisioned that one or more processor in communication one with the other, as well as various storage devices, may work singularly or in conjunction with one another to derive and execute pulse train schedules via, for example, a sunrise lamp apparatus comprising, perhaps, a pulse train emitter, various sensors and user interface devices and the like.

In accordance with some exemplary embodiments, aspects of the system may be adapted for application to one or more people in moving vehicles or methods of transport. In some embodiments, the entire cabin of the vehicle may be subjected to the same pulse train schedule. In other embodiments, sensors capturing data about individual passengers including, but not limited to thermal and visual imaging systems, may be utilized to provide individual pulse regimens whereby pulses are calibrated for each individual and delivered by aiming pulses directly at each individual. In one embodiment, the pulsing is administered by the individual overhead lamps in buses, trains, and airplanes. In some instances, pulse train therapy may be included in the ticket price or may be ordered individually, such as by providing a credit card, for provision during the flight. In one embodiment, each user in a group setting provides authorization to receive circadian phase shifting pulse trains.

In one embodiment, the secondary functionality and high EML light source use common circuitry 103. For example, referring back to the embodiment of FIG. 1, the system 100 shown is a sunrise lamp combined with a high EML light source 101 configured to emit pulsed light. The secondary functionality is a sunrise lamp system comprising the light sources 102 which subsume the high EML light source 101. More specifically, the light sources 102 include a full spectrum of light—i.e., long reds intermediate greens/yellow, and short blues and violets. Among the spectrum is the high EML light (e.g. 450-500 nm). Thus the high EML light source 101 is shared circuitry 103. Aside from the high EML light source 101, other shared circuitry shown system 100 of FIG. 1 includes the processor 110, memory 111, power supply 113 and driver 112.

These and other advantages maybe realized in accordance with the specific embodiments described as well as other variations. It is to be understood that the above description is intended to be illustrative, and not restrictive. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled.

What is claimed:

1. A system of adjusting the phase of a user's circadian rhythms, said system comprising:
a high EML light source configured for delivering pulsed high EML light in a pulsed mode of operation, wherein the pulsed high EML light has a frequency and intensity sufficient to effect circadian phase adjustment in said user; and
secondary functionality comprising transmitting electromagnetic radiation (EMR) to said user.

2. The system of claim 1, wherein the secondary functionality and the pulse trains use common circuitry.

3. The system of claim 2, wherein the secondary functionality involves light generation.

4. The system of claim 3, wherein the light generation involves light therapy.

5. The system of claim 4, wherein the light therapy involves a delivering high EML light to stimulate a user.

6. The system of claim 5, wherein said high EML light source and said secondary functionality are housed in a sunrise lamp.

7. The system of claim 1, further comprising:
one or more processing devices; and
a non-transitory memory device in communication with the one or more processing devices, the non-transitory memory storing instructions that when executed by the one or more processing devices, result in:
(a) receiving one or more sensor inputs indicative of at least one of a circadian rhythm of an individual, an environmental attribute, or a location attribute;
(b) determining based, at least in part, on the received one or more sensor inputs a pulse train schedule;
(c) emitting light from the high EML light source 101 at the user in accordance with the pulse train schedule, wherein the pulse train schedule includes the duration and time at which the pulsed EML light is delivered to a user to produce a predetermined shift in the phase of the user's circadian rhythm; and
(d) activating said secondary functionality.

8. The system of claim 7, wherein said secondary functionality is generating light to moderate circadian rhythms.

9. The system of claim 7, further comprising at least one sensor for providing said one or more sensor inputs.

10. The system of claim 9, wherein said at least one sensor comprises a wearable device.
